(19) United States
(12) Patent Application Publication
(10) Pub. No.: US 2008/0086105 A1
(43) Pub. Date: Apr. 10, 2008

(54) Protective underwear with abdominal support

(75) Inventor: Thanorm Sour, King of Prussia, PA (US)

Correspondence Address: Michael G. Panian;Covidien, 601 Allendale Road, King of Prussia, PA, 19406, US

(73) Assignee: Tyco Healthcare Retail Services AG, Neuhausen am Rheinfall (CH)

(21) Appl. No.: 11/903,234

(22) Filed: Sep. 21, 2007

Related U.S. Application Data
(60) Provisional application No. 60/849,421, filed on Oct. 4, 2006.

(51) Int. Cl. A61F 13/15

(52) U.S. Cl. 604/385.23; 604/385.01

(57) ABSTRACT

A protective undergarment having an absorbent chassis and an abdominal support member extending across the region of the undergarment which covers that abdomen of the wearer in order to provide abdominal support. The support member can be a pair of elastomeric panels which provide a supportive surface adjacent the abdomen of the wearer. The length of the panels can be adjustable to vary the degree of support, and the supportive feature thereof can also be provided by the stretch and support characteristics of the elastomeric material.

### CROSS-REFERENCE TO RELATED APPLICATIONS

This application claims priority from U.S. Provisional Application Ser. No. 60/849,421 entitled “Protective Underwear with Abdominal Support”, filed on Oct. 4, 2006, the entire content of which is hereby incorporated by reference and relied upon in its entirety.

### BACKGROUND

The protective undergarment with abdominal support relates generally to disposable absorbent undergarments, and more specifically to a disposable protective undergarment which also provides abdominal support.

Conventional protective undergarments do not provide abdominal support. The abdomen can become weak after surgery, child birth, hernias, and from illnesses which can cause frequent coughing and sneezing. Abdominal support garments are known in the art. One example of an undergarment to prevent abdominal and/or inguinal hernias from bulging while the garment is worn is disclosed in U.S. Pat. No. 4,059,103, to Glaser. Glaser describes a one-piece, approximately I-shaped fabric with a front and rear area connected by a narrow neck portion passing through the crotch. Tension on the supporting fabric is provided by elastic bands which are stitched to the front and rear sections and pass over the hip. It is disclosed that the garment, with a slight modification to accommodate anatomical differences, can equally serve males and females, and can be provided in basically two styles of the same design and structure, one style for one or two-sided hernias, and the other style for one-sided hernias only.

Another type of prior art abdominal support garment is described in U.S. Pat. No. 4,596,253, to Griffith. According to Griffith, as a result of either weakened abdominal muscles or an abundance of adipose tissue, some persons develop a distention of the lower abdomen. Such a distention is not only aesthetically unpleasing, but also the cause of physical discomfort. In women, the problem is particularly acute since the distention most frequently occurs in the lower area of the abdomen, i.e., below the waistline in the anterior wall adjacent the pelvic cavity. With the lower abdominal muscles weakened, the uterus, bladder, and other organs within the pelvic cavity fall from their normal anatomical position, causing pain and, often, menstrual difficulties. The discomfort resulting from distention of the abdomen is not confined solely to the pelvic region but may also occur in the lower back. Although not attributable necessarily to weakened abdominal muscles or excessive fat deposits, lower back pain is also a frequent problem for women during pregnancy.

In response, Griffith describes an abdominal support garment, particularly an undergarment for women, which provides the degree of correction needed to support the anterior abdominal wall and a cosmetic reshaping of the abdomen to yield a flat appearance. This garment is described as having a support panel and a shaping panel. The support panel has three elastic webs with their respective grains running at contrasting angles, wherein, during use, the support panel is positioned on the lower abdomen and the webs interact with one another so that the support panel assumes a pouch-like shape that holds and lifts the abdomen. The shaping panel is connected to the waistband and positioned in front of the support panel, and includes two elastic side webs that interact with a nonelastic central web to cause the shaping panel to assume a curvilinear operative configuration. A lower portion of the shaping panel conforms to the pouch-like shape of the support panel and cooperates therewith to hold and lift the abdomen, and an upper portion of the shaping panel remains substantially flat and operates against the abdomen to spread the same between the hipbones and provide a flat appearance.

Protective undergarments, for moderate-to-heavy incontinence needs, are also known in the art. One prior art protective undergarment is described in U.S. Pat. No. 6,478,786, to Glaug et al., which is hereby incorporated herein by reference, and which is assigned to the assignee of the present application. Glaug et al. describes a protective and, e.g., disposable, underwear arranged to be worn by a person to trap and collect loose or liquid waste products. This underwear can comprise a pants-shaped chassis having a waist portion, a crotch portion and a pair of leg openings disposed on opposite sides of the chassis. The crotch portion is located between the leg openings and has a front section and a rear section. The waist portion has a front section and a rear section joined together at the sides of the chassis. The front and rear sections of the crotch portion merge together at the bottom of the chassis and are located opposite to the waist portion. Each of the leg openings has an arcuate upper section and an arcuate lower section. The chassis can comprise a hydrophobic or hydrophillic, e.g., a spunbond/meltblown/spunbond, non-woven material. The underwear can further comprise an insert comprising an insert sheet, a liquid permeable, e.g., a spunbond polypropylene, non-woven material, a liquid absorbent core, e.g., pulp and super absorbent particulates disposed between a fluid acquisition layer and the liquid impermeable film, e.g., polyethylene film, and the heretofore mentioned fluid acquisition layer, e.g., a thru-air-bonded bicomponent fiber with a fast finish surfactant, between the core and the liquid permeable non-woven material insert sheet. The fluid acquisition layer may be eliminated, if desired. The insert may further comprise a liquid impermeable film, e.g., polyethylene film, located between the core and the chassis. The waist portion of the chassis can have plural transversely oriented elastic threads extending parallel to one another. These plural transversely oriented elastic threads can encompass a substantial portion of the chassis from the waist portion to a point adjacent the crotch portion. The chassis may also comprise a pair of longitudinally oriented elastic threads intersecting a least one of the transversely oriented elastic threads of the front section of the waist portion and intersecting at least one of the transversely oriented elastic threads of the rear section of the waist portion to form an enclosed compartment for the liquid absorbent core to prevent the egress of liquid therefrom. The upper section of each of the leg openings is located adjacent the waist portion and can be elasticized by at least one arcuate elastic thread. Portions of the longitudinally oriented elastic threads are located very close to the lower sections of the leg openings to elasticize the lower sections of the leg openings, whereupon the leg openings closely conform about the leg of the person.

The above-described prior art undergarments provide examples of both abdominal support undergarments and protective undergarments, but none provide the benefits of both incorporated into a single undergarment. Accordingly, there is a need for a protective undergarment with abdominal support for persons that may have a need for both incontinence protection and support for weak or injured abdominal muscles.

### SUMMARY

A protective undergarment with abdominal support is provided which can have an absorbent chassis and an abdominal support member extending across the abdomen of the wearer. The absorbent chassis can have a front region, a rear region, and a crotch region extending therebetween. The abdominal support member can be attachable across the front region to provide the desired degree of abdominal support. Such a protective undergarment can both provide protection for moderate-to-heavy incontinence needs and support for injured or weakened abdominal muscles. The support member can be added onto, or could be made integral with, the protective undergarment. The support member can be incorporated into the protective undergarment by, for example, affixing opposite sides of the support member to respective side seams of the protective undergarment, with the support member crossing over the front region of the protective undergarment which covers abdominal area of the person wearing the undergarment. The support member can be, in whole or part, a smooth front panel of elastomeric material, and can be designed to have an adjustable length, such that it could be tightened or loosened in order to vary the degree of support provided. The degree of elasticity of the elastomeric material can also factor into the amount of support provided by the panel. Moreover, the front panel could alternatively have a fixed length, such that the elasticity of the elastomeric material could generally determine the amount of support provided.

Additional objects and advantages will become apparent in light of the attached drawing and the following description of the protective garment with abdominal support.

### BRIEF DESCRIPTION OF THE DRAWING FIGURES

A more complete understanding of the protective garment with abdominal support described herein can be obtained by considering the following description in conjunction with the accompanying drawing figures.

FIG. 1 illustrates a front view of an embodiment of a protective undergarment with abdominal support.

FIG. 2 illustrates a front view of another embodiment of a protective undergarment with abdominal support.

### DESCRIPTION OF CERTAIN EMBODIMENTS

A protective undergarment with abdominal support can comprise an absorbent article and an abdominal support member extending over the abdomen of the wearer, such that the undergarment can provide both protection for incontinence needs and support for injured or weakened abdominal muscles.

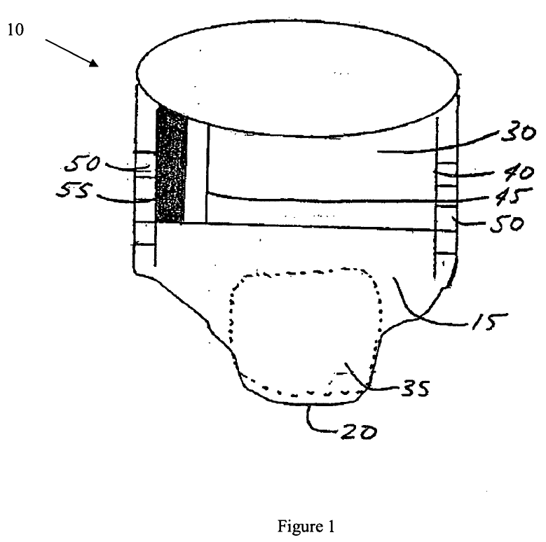

Referring now to the drawing figures, FIG. 1 illustrates a front view of an embodiment of a protective undergarment with abdominal support, which can generally comprise a chassis 10 having a front region 15, a rear region (not shown), a crotch region 20 extending therebetween, and an abdominal support member 30 attachable across the abdomen of the wearer. The chassis 10 can further comprise a liquid-permeable topsheet (not shown), a liquid-impermeable backsheet (not shown), and a liquid-absorbent core 35 disposed at least partially therebetween. The abdominal support member 30 can comprise a front panel having a fixed end 40 attached to the chassis 10 and a free end 45 that can be extendable across the front region 15. The fixed end 40 can be attached in some manner to the chassis 10, such as, for example, a side seam 50 of the protective undergarment. The free end 45 of the front panel can be configured for attachment across the front region 15 of the chassis 10 which covers the abdominal region of the wearer by a fastener 55. The fixed end 40 and/or free end 45 can be releasably attachable to the chassis 10 in order to provide the desired degree of abdominal support.

The abdominal support member 30 can be added onto, or made integral with, the protective undergarment by, for example, affixing opposite ends of the support member 30 to respective side seams 50 of the protective undergarment, with the abdominal support member 30 extending over the abdominal region. The abdominal support member 30 could be, in whole or part, a front panel of elastomeric material, and can be designed to have an adjustable length, such that it could be tightened or loosened in order to vary the degree of support provided. The degree of elasticity of the elastomeric material can also factor into the amount of support provided by the panel. The panel could have a fixed length, such that the elasticity of the elastomeric material would generally determine the amount of support provided. The front panel could be made substantially, or only partly, from a smooth elastomeric material.

In alternative embodiments, the protective undergarment with abdominal support can comprise a protective undergarment or other incontinence garments for infants, children, and adults, including disposable diapers wherein the front and rear regions of the chassis 10 are joined along a pair of opposed side seams 50, disposable pull-on diapers, disposable training pants, disposable underwear, and underwear with disposable absorbent pads.

The manner of attachment of the free ends 45 of the front panels can be designed to provide a range of adjustment so as to permit customizing the desired amount of support. The manner of attachment can be provided by a fastener 55, for example, via hook and loop fastener portions at cooperating free ends of the front panels. However, it will be understood by those of ordinary skill in the art that other methods of attachment, for example, mechanical fasteners, adhesive tapes, tab fasteners, mushroom fasteners, snaps, pins, buttons, belts, buckles, and the like, could be utilized to similarly provide for varying degrees of adjustability to customize the amount of support provided. Additionally, it will further be recognized that the free ends 45 could be attached to each other in a non-adjustable manner, and the fixed ends 40 could be attached to the side seams 50 of the undergarment in manner to provide adjustability so as to permit the same varying degrees of support. Alternatively, or additionally, the length of one or both front panels could be made adjustable to provide the adjustability feature.

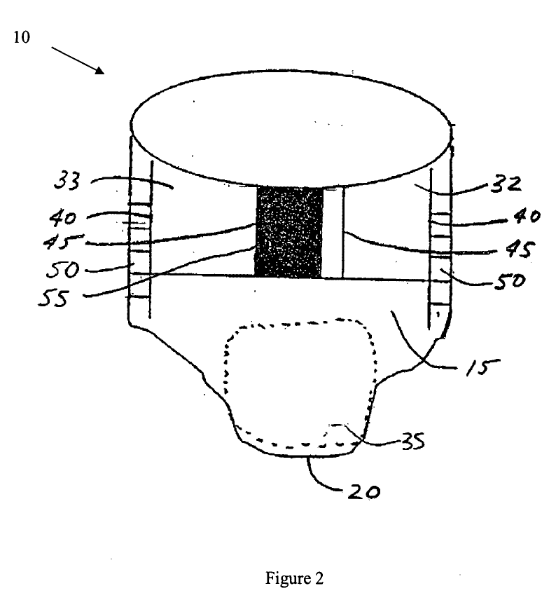

Another embodiment of the abdominal support member 30 is illustrated in FIG. 2, which can generally comprise a left front panel 32 and a right front panel 33, each having a fixed end 40 attached to the chassis 10 and a free end 45 that can be extendable across the front region 15. One or both front panels 32, 33 could be made substantially, or only partly, from a smooth elastomeric material. One or both of the front panels 32, 33 could have a fixed or adjustable length. The fixed end 40 of each front panel 32, 33 can be attached in some manner to the chassis 10, such as, for example, to respective left and right side seams 50 of the chassis 10. The free end 45 of each front panel 32, 33 can be configured for attachment to each other or an opposite side seam 50 across the front region 15 of the chassis 10 which covers the abdominal region of the wearer. The embodiment depicted in FIG. 2 can otherwise function in a similar manner as described previously in connection with the embodiment depicted in FIG. 1.

Although certain embodiments of the invention have been described in detail, it will be appreciated by those skilled in the art that various modifications to those details could be developed in light of the overall teaching of the disclosure. Accordingly, the particular embodiments disclosed herein are intended to be illustrative only and not limiting to the scope of the invention.

What is claimed is:

1. A protective undergarment with abdominal support comprising:
a chassis having a front region, a rear region, and a crotch region extending therebetween; and
an abdominal support member attachable across said front region to provide a desired degree of abdominal support.

2. The protective undergarment with abdominal support of claim 1 wherein said abdominal support member is adjustable to provide varying degrees of abdominal support.

3. The protective undergarment with abdominal support of claim 1 wherein said abdominal support member further comprises:
a front panel of elastomeric material having a first end and a second end;
said first end attached to said chassis;
said front panel extendable across said front region of said chassis; and
at least one of said first and seconds releasably attachable to said chassis to provide the desired degree of abdominal support.

4. The protective undergarment with abdominal support of claims 3 wherein said front and rear regions are joined along a pair of opposed side seams.

5. The protective undergarment with abdominal support of claims 3 wherein said attachment is selected from the group consisting of mechanical fasteners, adhesive tapes, tab fasteners, hook and loop fasteners, mushroom fasteners, snaps, pins, buttons, belts, buckles, and combinations thereof.

6. The protective undergarment with abdominal support of claim 1 wherein said abdominal support member further comprises:
a pair of abdominal support members;
each of said pair of abdominal support members having a first end and a second end;
each of said first ends of said pair of abdominal support members attached to said chassis;
each of said second ends of said pair of abdominal support members extendable across said front region of said chassis; and
each of said second ends of said pair of abdominal support members attachable to at least one of each other and said chassis to provide the desired degree of abdominal support.

7. The protective undergarment with abdominal support of claims 6 wherein said attachment is selected from the group consisting of mechanical fasteners, adhesive tapes, tab fasteners, hook and loop fasteners, mushroom fasteners, snaps, pins, buttons, belts, buckles, and combinations thereof.

8. The protective undergarment with abdominal support of claims 6 wherein said front and rear regions are joined along a pair of opposed side seams.

9. The protective undergarment with abdominal support of claim 8 wherein at least one of said first ends is joined to one of said pair of side seams.

10. The protective undergarment with abdominal support of claim 1 wherein said abdominal support member comprises a smooth front panel of elastomeric material.

11. The protective undergarment with abdominal support of claim 1 wherein said abdominal support member comprises an integral portion of said chassis.

12. The absorbent chassis of claim 1 wherein said absorbent chassis further comprises:
a liquid-permeable topsheet;
a liquid-impermeable backsheet; and
a liquid-absorbent core disposed therebetween.
